(19) United States
(12) Patent Application Publication
(10) Pub. No.: US 2006/0264786 A1
(43) Pub. Date: Nov. 23, 2006

(54) Method and system for analyzing status of balance functions in patients with acute neurological disorders

(76) Inventor: Lewis M. Nashner, Portland, OR (US)

Correspondence Address: BROMBERG & SUNSTEIN LLP, 125 SUMMER STREET, BOSTON, MA, 02110-1618, US

(21) Appl. No.: 11/134,834

(22) Filed: May 20, 2005

(51) Int. Cl. A61B 5/00; A61B 5/05; A61B 5/103

(52) U.S. Cl. 600/595; 600/587; 600/552; 600/554

(57) ABSTRACT

A method for assessing a subject's ability to sense orientation using somatosensory inputs derived from contact of the subject with a support surface includes, while partially assisting the subject in bearing the subject's weight on the support surface, performing a protocol. The protocol includes tilting the support surface away from an actual horizontal position in a given direction and having the subject cause repositioning of the support surface until it is in a perceived horizontal position. The perceived horizontal position is compared with the actual horizontal position.

### TECHNICAL FIELD

The present invention relates to neurological disorders and, in particular, to methods and systems for analyzing the status of balance functions in subjects with acute neurological disorders.

### BACKGROUND ART

Following an acute neurological injury such as a stroke, subjects frequently experience moderate to severe sensory and motor system disorders that render them unable to stand freely for extended periods of time and/or unable to walk. Although these deficits can be very debilitating in the acute phase, a reported two-thirds of stroke patients eventually regain some level of independent mobility with or without the use of assistive devices such as canes or walkers (See Jorgensen H S, Nakayama H, Raaschou H O. Neurologic and functional recovery. The Copenhagen stroke study. Physical Medicine and Rehabilitation: Clinics of North America 10:887-906. 1999, which is hereby incorporated herein by reference). The key to recovery is an appropriately designed rehabilitation management program, requiring an average three months to complete. (See Bogey R A, Geis C C, Bryant P R, Moroz A, O'Neill B J. Stroke and neurodegenerative disorders. 3 Stroke rehabilitation management. Archives of Physical Medicine and Rehabilitation 85, Supplement 1. 2004, which is hereby incorporated herein by reference).

Care providers are faced with several challenges during the acute phase of a subject's illness. These include: (1) determining the rehabilitation program most appropriate for the individual patient and (2) counseling the patient and family regarding the necessary type and duration of treatment and the probable extent of functional recovery. Objective information enabling providers to more effectively plan treatment and more accurately predict the time course and extent of recovery would be of significant value in the acute planning phases of care. In a review of clinical literature related to recovery of mobility function after stroke, researchers identified five impairments that were consistently correlated with gait performance. Balance was identified as one of the most influential impairments among the five. (See Bohannon R W, Andrews A W. Relationship between impairments and gait performance after stroke: a summary of relevant research. Gait & Posture 3:236-240. 1995, which is hereby incorporated herein by reference). Hence, status of the balance system following a stroke is considered one of the important prognostic indicators for the likely extent of recovery. (See Brosseau L, Philippe P, Potvin L, Boulanger Y-L. Post stroke inpatient rehabilitation 1. Predicting length of stay. American Journal of Physical Medicine and Rehabilitation 75:422-430, 1996, which is hereby incorporated herein by reference.)

It is generally understood that balance function relies on information from three senses; including, the vestibular system, somatosensory information derived primarily from the forces and motions of the feet in contact with the support surface, and vision. It is further understood that in adult subjects, somatosensory information tends to be the dominant sense. Among the three sensory systems for balance, there are adequate means described in the prior art for assessing vestibular and visual system functions without requiring subjects to stand freely. In contrast, prior art means for assessing somatosensory functions of the feet and legs as they relate to automatic mechanisms for balance control are limited to means requiring that the subject stand freely. This is because automatic mechanisms for balance control tend to be suppressed when subjects are provided with additional support during standing.

Recent research studies have demonstrated that the efficacy of somatosensory inputs to balance control may be enhanced by vibrating the support surface on which a subject stands freely or by applying a direct vibratory stimulus to one or more muscles of a standing subject's legs (see for example Priplata A A, Niemi J B, Lipsitz L A, Collins J J. Vibrating insolesand balance control in elderly people. The Lancete 362:1123-24. 2003, which is herein incorporated by reference.) It is generally believed that vibration enhances somatosensory inputs at the level of the peripheral sensors. Thus, in a post-stroke subject whose somatosensory loss is due to impairment of the brain's ability to process somatosensory information, vibration may or may not improve balance function. As an alternative to the mechanical stimulus provided by vibration, somatosensory inputs from an extremity may also be enhanced by means of small constant or periodically varying electrical currents applied to one or more muscles or to one or more nerves innervating the muscles. Such currents may be applied through surface electrodes placed over the targeted muscles and/or nerves or through needle electrodes inserted into the targeted muscles and/or nerves.

Numerous devices and methods have been described in the prior art for assessing balance functions of free standing subjects. One class of such devices monitor the spontaneous swaying motions of individuals freely standing on a support surface. The means employed for quantifying sway motions include force sensing surfaces on which the individual stands (one example being a Accusway^{Plus} system manufactured by AMTI, Watertown, Mass.), body motion sensing technologies including accelerometers and video motion analyzers (one example being the 3-D optical motion capture system manufactured by MotionAnalysis Corporation, Santa Rosa, Calif.). A second class of devices includes those in which a support surface on which the subject stands as well as the visual surround are capable of moving while a standing individual's sway is monitored. One such device, named EquiTest® and manufactured by Neurocom International of Clackamas, Oreg., includes supporting and visual surround surfaces under independent servo-control by a computer. Independent servo-control of the support and visual surround surface enable the EquiTest® system to isolate and quantify the individual contributions of vestibular system, somatosensory, and visual inputs to balance, and to quantify the efficacy of the automatic response mechanisms controlling balance. Methods pertaining to this system are described in U.S. Pat. Nos. 4,738,269, 4,830,024, 5,052,406, 5,269,318, 5,303,715, and 5,551,445 (all of which are hereby incorporated herein by reference). Several other devices employing passive compliant support surfaces include the BalanceQuest manufactured by Micromedical Technologies, Inc. and the Stability System manufactured by Biodex Medical Systems, Shirley, N.Y.

A third class of devices include those that monitor locomotive activities; including mobility tasks such as walking in a straight line, turning, getting in and out of a chair, reaching, and climbing up or down a step. Devices in this class employ force sensing surfaces (such as, for example, the Balance Master, manufactured by Neurocom International Inc.) Assessments using such devices are described in U.S. Pat. Nos. 5,474,087, 5,476,103, 5,623,944 and 6,010,465 (all of which are hereby incorporated herein by reference). One example of a gait measuring device using accelerometer technology is the LifeGait system manufactured by MiniSun LLC, Fresno, Calif.

Common among many of the above described balance assessing means is the requirement that the individual being assessed must be standing freely and/or freely moving. Restraining or otherwise supporting individuals during these types of assessment may either partially or completely defeat the purpose of quantifying the individual's balance impairment. The is because critical elements of balance function are inhibited whenever a standing individual is provided with alternative means for maintaining balance; including such alternatives as touching or grasping a support rail with the hand and being suspended from a supporting harness. (See Dichgans J, Held R, Young L, Brandt T. Moving visual scenes influence the apparent direction of gravity. Science 178:1217-1219. 1972 (which is hereby incorporated herein by reference).

In the acute phase following a stroke many patients are either completely incapable of standing without external support or are capable of doing so only for very brief periods of time. Hence, devices based on free stance for extended periods of time are ineffective in assessing a significant percentage of stroke patients in the acute phase.

A class of devices described in the prior research art assesses an individuals' ability to sense orientation relative to the gravitational vertical without requiring free stance. In one embodiment, termed the subjective visual vertical test, the individual sits in a completely dark room and views an illuminated object that is tilted away from the gravitational vertical by a degree unknown to the viewing individual. The individual is given the means to manipulate the orientation of the illuminated object and instructed to reposition the object until it appears vertical. The orientation selected by the individual is recorded and compared to the actual vertical to determine the accuracy of the individual's perceived vertical. (See Mittelestadt H. A new solution to the problem of the subjective vertical. Naturwissenschaften 70:272-281. 1983, Miller E F, Fregly A R, Graybiel A. Visual horizontal-perception in relation to otolith function. American J of Psychology 81:488-496. 1968 and Bisdorff A, Bronstein A, Gresty M, Wolsley C. Subjective postural vertical inferredfrom vestibular-optokinetc vs. proprioceptive cues. Brain Research Bulletin 40:413-415. 1996, all of which are hereby incorporated herein by reference.)

In another embodiment described in the prior art, the individual is seated in a chair that is tilted away from the vertical position and placed within a completely dark room. Seated subjects are provided with means to control the orientation of the chair and instructed to reposition the chair until it is perceived to be in a vertical position relative to gravity. (See Bisdorff A, Bronstein A, Gresty M, Wolsley C. Subjective postural vertical inferred from vestibular-optokinetc vs. proprioceptive cues. Brain Research Bulletin 40:413-415. 1996, which is hereby incorporated herein by reference.) Again, chair positions were recorded to quantify the individual's perceived postural vertical. Various protocols employing visual, inner ear, and tactile stimulation were then imposed to quantify the influences of these senses on perceived vertical. Investigators employed the above methods to assess patients with vestibular balance disorders. (See Bisdorff A, Wolsley C, Anastasopoulos D, Bronstein A, Gresty M. The perception of body verticality (subjective postural vertical) in peripheral and central vestibular disorders. Brain 119:1523-1534. 1996.

While the above described devices and methods for quantifying an individual's perceived visual and seated postural verticals meet the requirement of assessing an element of the balance system without requiring free stance, they do not meet the requirement of quantifying balance function related to the free standing and walking for the following reason. The prior art embodiments with individuals seated and required to reposition the seat assess the individual's perceptions related to combined inputs of the vestibular system and the forces and motions of contact between the buttocks and lower trunk and the seat. Prior art embodiments requiring patients to reposition a visual object assess perceptions related primarily to the vestibular system. It is generally accepted in the prior medical art that somatosensory information related to the forces and motions of contact between the feet and legs and the support surface provide critical orientation information to the systems responsible for maintaining balance during free standing and walking. It is also generally accepted that different areas of the brain are involved in sensing and controlling muscles and joints of the legs, arms, and trunkal portions of the body and hence that the distribution of sensory and motor losses vary among stroke patients, depending on the specific areas of the brain affected by the disease. As a practical consequence, evaluating a seated patient's perceptions of orientation may not provide information related to their ability to correctly perceive orientation based on somatosensory inputs while in freely standing and walking positions.

An additional problem is that, in the individual stroke patient, the distribution of sensory and motor deficits typically differs significantly between the left and right sides of the body. Since it is not possible to mechanically isolate forces and motions of the left and right sides of a seated individual's buttocks and trunkal body parts, assessments based on the seated position have the further disadvantage of not allowing independent assessment of left and right side sensory functions. Hence, the patient with normal somatosensory inputs on one side of the body and impaired inputs on the other may perform normally when receiving adequate information from the unimpaired side of the body as a consequence of standing on both legs simultaneously, thereby obscuring the presence of impaired somatosensory input on the other side.

### SUMMARY OF THE INVENTION

In a first embodiment of the invention there is provided a method for assessing a subject's ability to sense orientation using somatosensory inputs derived from contact of the subject with a tiltable support surface. The method includes, while partially assisting the subject in bearing the subject's weight on the support surface, performing a protocol. The protocol includes tilting the support surface away from an actual horizontal position in a given direction and having the subject cause repositioning of the support surface until it is in a perceived horizontal position. The perceived horizontal position is compared with the actual horizontal position.

In accordance with related embodiments of the invention, partially assisting the subject in bearing the subject's weight may include assisting the subject in achieving or maintaining a standing position on the support surface and/or assisting the subject in achieving and/or maintaining a sitting position on the support surface. In accordance with another related embodiment, the percentage of the total weight born by the subject is varied. In accordance with a further related embodiment, the method may include permitting the subject to utilize a stabilizing facility to achieve postural stability.

In accordance with other related embodiments, tilting the support surface away from the actual horizontal position may include tilting the support surface along a lateral axis such that the subject's toes are directed upward and/or tilting the support surface along a lateral axis such that the subject's toes are directed downward. In accordance with further related embodiments, tilting the support surface away from the actual horizontal position may include tilting the support surface along a forward directed axis such that the left side of the support surface is directed upward, tilting the support surface along a forward directed axis such that the right side of the support surface is directed upward and/or tilting the support surface along an axis other than a lateral or forward facing axis.

In accordance with still further related embodiments, the method may further include performing the protocol another time while applying a stimulus to one or both of the subject's legs when having the subject cause repositioning of the support surface, and comparing the actual horizontal position with the horizontal position as it is perceived by the subject, and comparing results obtained in performing the protocol with and without the stimulus. Applying a stimulus to one or both of the subject's legs may include applying a vibratory stimulus and/or applying an electrical stimulus.

In accordance with an additional related embodiment, tilting the support surface away from the actual horizontal position includes tilting the support surface along a lateral axis such that the subject's toes are directed upward, performing the protocol another time while tilting the support surface along a lateral axis such that the subject's toes are directed downward and comparing results obtained in performing the protocol with the subject's toes directed upward to results obtained in performing the protocol with the subject's toes directed downward.

In accordance with yet a further related embodiment, tilting the support surface away from the actual horizontal position includes tilting the support surface along a lateral axis such that the subject's toes are directed upward, performing the protocol another time while tilting the support surface along a forward directed axis such that the left side of the support surface is directed upward and comparing results of the obtained in performing the protocol with the subject's toes directed downward to results obtained in performing the protocol with the left side of the support surface directed upward.

In accordance with another related embodiment, tilting the support surface away from the actual horizontal position includes tilting the support surface along a lateral axis such that the subject's toes are directed upward, performing the protocol another time while tilting the support surface along a forward directed axis such that the right side of the support surface is directed upward and comparing results of the obtained in performing the protocol with the subject's toes directed downward to results obtained in performing the protocol with the right side of the support surface directed upward.

In accordance with yet a further related embodiment, tilting the support surface away from the actual horizontal position includes tilting the support surface along a lateral axis such that the subject's toes are directed upward, performing the protocol another time while tilting the support surface along an axis other than a lateral or forward facing axis and comparing results obtained in performing the protocol with the subject's toes directed downward to results obtained in performing the protocol with the support surface tilted along an axis other than a lateral or forward facing axis. In accordance with other related embodiment, the method also includes comparing the results of the protocol with results of the protocol performed by a defined population of control subjects.

In accordance with another embodiment of the invention, a system for assessing a subject's ability to sense orientation using somatosensory inputs derived from contact of the subject with a support surface includes a tiltable support surface, a harness system for partially assisting the subject in bearing the subject's weight on the support surface and a computational device coupled to the support surface for controlling the motion of the support surface and comparing an actual horizontal position with a perceived horizontal position to produce a first result.

In accordance with related embodiments, the computational device may compare the first result with a second result derived from a defined population of control subjects. In accordance with another related embodiment, the system may also include a stimulator for applying stimulus to the subject. The stimulator may include a vibrator for applying a vibratory stimulus and/or the stimulator may include a electrical stimulator for applying an electrical stimulus to the subject. The stimulator may also be coupled to the computational device. In accordance with yet further related embodiments, the system may also include a stabilizing facility, graspable or touchable by the subject, for providing the subject with postural stability and/or a display device coupled to the computational device.

### BRIEF DESCRIPTION OF THE DRAWINGS

The foregoing features of the invention will be more readily understood by reference to the following detailed description, taken with reference to the accompanying drawings, in which:

FIG. 1 is a block diagram of a system, in accordance with an embodiment of the invention, for assessing a subject's ability to sense orientation using somatosensory inputs derived from the muscles and joints of at least one leg;

FIG. 2 is an illustration of the system of FIG. 1 showing a subject in an initial position wherein the support surface is titled;

FIG. 3 is an illustration of the system of FIG. 1 showing a subject in a final position wherein the support surface is in a level position;

FIG. 4 is a flow chart illustrating a method, in accordance with another embodiment of the invention, for assessing a subject's ability to sense orientation using somatosensory inputs derived from the muscles and joints of at least one leg; and

FIG. 5 is a flow chart illustrating a method, in accordance with a further embodiment of the invention, for determining the extent to which a subject's ability to perceive the position of a support surface is limited by the ability of the brain to process somatosensory inputs and/or the ability of somatosensory receptors to sense position.

### DETAILED DESCRIPTION OF SPECIFIC EMBODIMENTS

Embodiments of the present invention provide an objective, quantitative means to assess an individual's ability to sense orientation using somatosensory inputs derived from contact of the individual with a support surface while not requiring the individual to stand freely during test administration. In a typical embodiment, the somatosensory inputs are derived from the muscles and joints of a single leg or of both legs together, while the erect standing subject is suspended in a partial weight bearing harness while in contact with the support surface and bearing a fraction of the body weight. Such embodiments are pertinent in cases where the individual must be assisted in order to achieve or maintain a standing, sitting or other erect position.

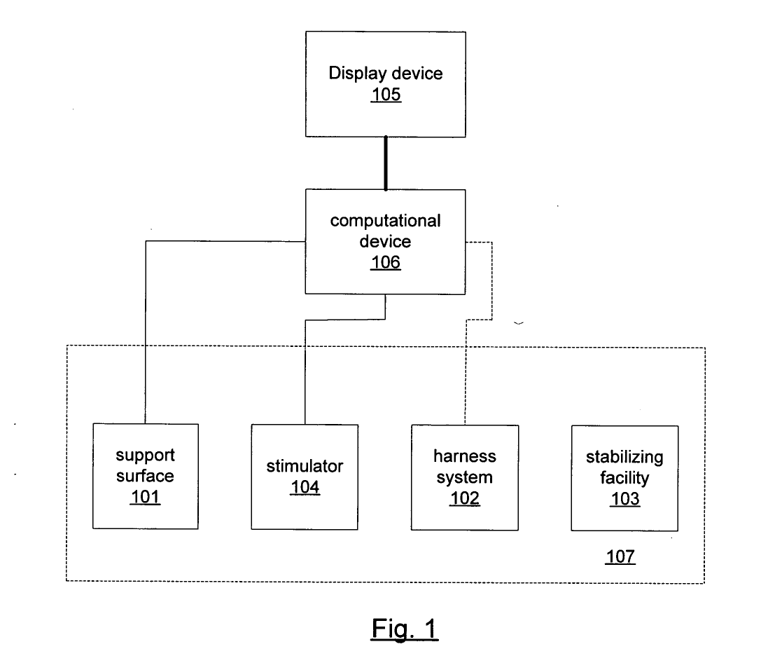

FIG. 1 is a block diagram illustrating of a system, in accordance with one embodiment of the invention, for assessing a subject's ability to sense orientation using somatosensory inputs derived from the muscles and joints of one or more extremities. In this embodiment, the somatosensory inputs are derived from the muscles and joints of at least one leg; however, somatosensory inputs may be derived from other parts of a subject's body such in accordance with the invention. The system includes a tiltable support surface 101, such as one or more force plates, a treadmill, a foam pad or a cushion. The system may also include a stimulator 104 for applying a stimulus to the at least one leg. The stimulator may be a vibrator for providing a vibratory stimulus or an electrical stimulator for applying an electrical stimulus. The system further includes a computational device 106, which may be a general purpose computer or microprocessor running a control process for controlling the motion of the support surface and the application of the vibratory or electrical stimulus as well as an analysis process for analyzing the results of one or more assessments. The control process may be coupled to an input device such as a keyboard, mouse or hardwired or wireless keypad. Alternatively, control process may be implemented in a dedicated controller that is operated by the subject or a test administrator. The system may further include a display device 105 for displaying information to the subject and/or displaying information related to an assessment to a test administrator. The system may also include a stabilizing facility 103 for use by the subject in achieving postural stability under a test protocol such as the protocol described below. Examples of such a stabilizing facility include one or more handrails or straps, which may be grasped or touched by the subject to assist the subject in achieving postural stability. A harness system 102 may also be provided for bearing a controllable amount of the weight of the subject for use under a test protocol such as the protocol described below. Together the support surface 101, the stabilizing facility 103 and the harness system 102 constitute a subject contact module 107 that is the portion of the system in contact with the subject. The stabilizing facility 103 and the harness system 102 may be used selectively (that is, with some subjects only one of them may be used) or in cooperation with one another (that is, used together). In one embodiment, the stabilizing facility 103 and the harness system 102 are implemented by a Biodex Unweighing System, available from Biodex Medical Systems, Inc. of Shirley, N.Y.)

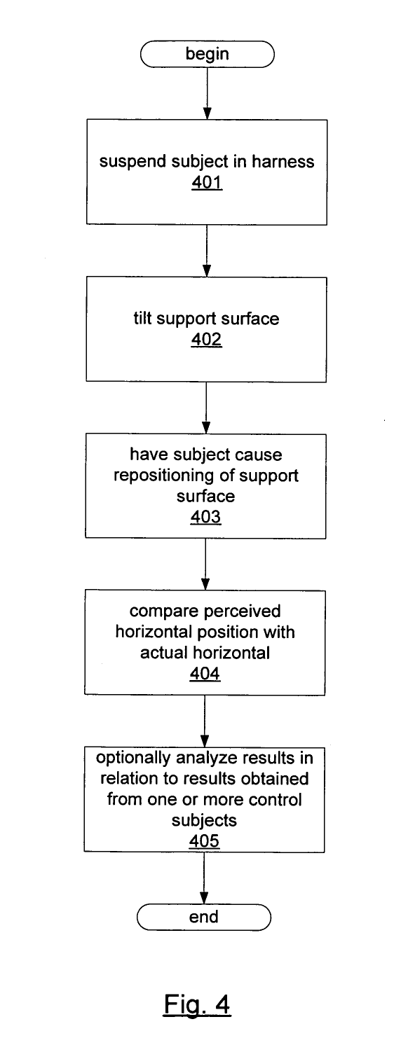

FIG. 4 is a flow chart illustrating a method for assessing a subject's ability to sense orientation using somatosensory inputs derived from the muscles and joints of at least one leg in accordance with an embodiment of the invention. In accordance with this embodiment, the subject is suspended, in process 401, in an erect position within an overhead harness system with at least one foot placed on a tiltable support surface. The harness system provides postural stability and bears a percentage of the subject's total body weight that is equal to or greater than 0% of the total body weight but less than 100%. Because the harness does on support the total weight of the subject, contact forces still exist between the contacting foot and the support surface.

The percentage of body weight born by the harness can be varied, depending on the ability of the individual subject to bear weight. Either one or both feet can be in contact with the support surface. The subject may also stand on the support surface (with either one or both legs) and the subject may be allowed to touch or grasp handrails. While not bearing a significant percentage of body weight, the handrails provide the subject with enhanced postural stability.

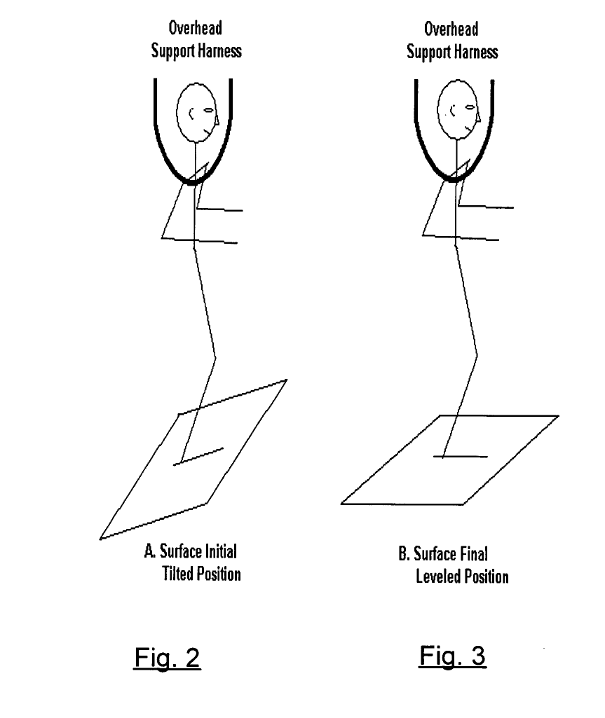

The support surface is tilted, in process 402, away from an actual horizontal in a given direction (for example, toes up, toes down, left side up, right side up, or in a diagonal direction) as shown in FIG. 2. The initial extent of tilt is sufficiently great for the subject to sense the surface tilt, even when deprived of any visual reference to the surface. Without the benefit of visual information, the subject is instructed, in process 403, to reposition the support surface until it is in a perceived horizontal position as shown in FIG. 3. To accomplish this part of the test, the subject may be provided with an arrangement for directly controlling the support surface orientation as described above, or alternatively, may instruct an operator who is in control of the support surface orientation. When the subject indicates that the surface orientation is perceived as horizontal, the perceived horizontal orientation is recorded and compared, in process 404, to the actual horizontal position. The foregoing procedure may be repeated using different surface starting positions, different percentages of body weight, and different combinations of single and double leg standing.

Following completion of tests described in the preceding paragraph, results may compared and/or analyzed, in process 405, in accordance with either one or both of the following two methods. First, to determine whether or not a subject's ability to sense surface position relative to a given leg, starting position, and body weight bearing percentage is within a range likely to permit recovery of balance and mobility function, the subject's results may be compared, using statistical tests for determining measurement differences well known in the prior art, to the results achieved in the same tests by subjects within a defined control population. Examples of control populations of subjects may include those with no known balance system deficits, those who suffered neurological deficits but later recovered their balance and mobility functions, and those who suffered neurological deficits and later failed to recover their balance and mobility functions. Second, additional analyses may identify asymmetries in the subject's ability to sense surface position with the left and right legs, or for each leg separately or for the two legs together as well as asymmetries in ability to sense position in the left and right or the forward and backward directions. To determine the significance of leg and directional asymmetries, differences in results may be compared, again using statistical tests for determining measurement differences well known in the prior art, to comparable difference results achieved by subjects within one or more control populations. Examples of control populations of subjects include those with no known balance system deficits, those who suffered neurological deficits and later recovered their balance and mobility functions, and those who suffered neurological deficits and later failed to recover their balance and mobility functions.

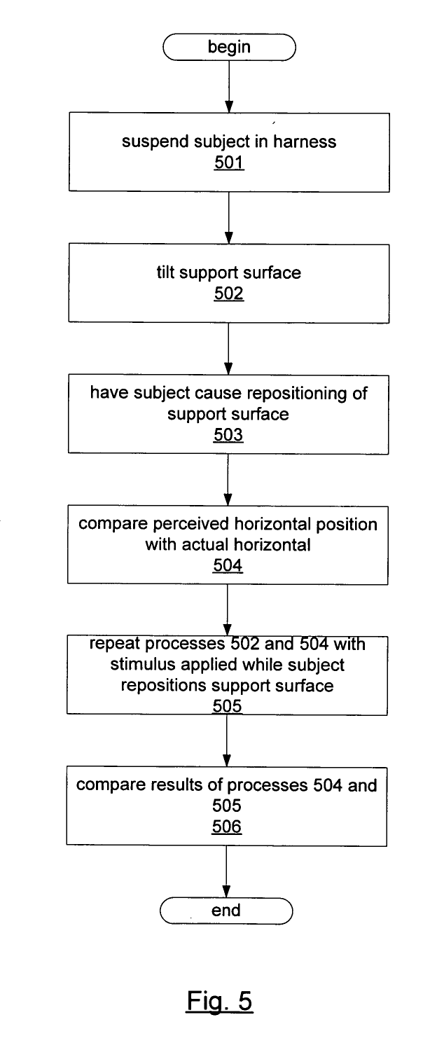

FIG. 5 is a flow chart illustrating a method, in accordance with a further embodiment of the invention, for determining the extent to which a subject's ability to perceive the position of a support surface is limited by the ability of the brain to process somatosensory inputs and/or the ability of somatosensory receptors to sense position. To determine the extent to which the subject's ability to perceive the position of the support surface is limited by either one or both of (1) the ability of the brain to process somatosensory inputs or (2) the ability of somatosensory receptors and peripheral nerves from either one or both logs to sense position relative to the support surface, the subject is suspended, in process 501, in an erect position within an overhead harness system with at least one foot placed on a tiltable support surface as described above. The support surface is tilted, in process 502, away from an actual horizontal in a given direction. Again, without the benefit of visual information, the subject is instructed, in process 503, to reposition the support surface until the subject perceives it to be in the horizontal position. When the subject indicates that the surface orientation is perceived as horizontal, the perceived horizontal orientation is recorded and compared, in process 504, to the actual horizontal position. Processes 502 through 504 described above are repeated, in process 505, with vibration applied to either one or both of the legs in contact with the support surface while the subject repositions the support surface. Results of the tests performed with and without vibrations in one or both legs are then compared, in process 506, to quantify differences in the subject's perception of horizontal in each leg separately and the two legs together with and without the presence of vibration.

While the invention has been described in connection with specific embodiments thereof, it will be understood that it is capable of further modification. This application is intended to cover any variation, uses, or adaptations of the invention and including such departures from the present disclosure as come within known or customary practice in the art to which invention pertains.

What is claimed is:

1. A method for assessing a subject's ability to sense orientation using somatosensory inputs derived from contact of the subject with a tiltable support surface, the method comprising:
while partially assisting the subject in bearing the subject's weight on the support surface,
performing a protocol that includes:
tilting the support surface away from an actual horizontal position in a given direction;
having the subject cause repositioning of the support surface until it is in a perceived horizontal position; and
comparing the perceived horizontal position with the actual horizontal position.

2. A method according to claim 1, wherein partially assisting the subject in bearing the subject's weight includes assisting the subject in maintaining a standing position on the support surface.

3. A method according to claim 1, wherein partially assisting the subject in bearing the subject's weight includes assisting the subject in maintaining a sitting position on the support surface.

4. A method according to any of claims 1-3, wherein the percentage of the total weight born by the subject is varied.

5. A method according any of claims 1-3, further comprising:
permitting the subject to utilize a stabilizing facility to achieve postural stability.

6. A method according to claim 2, wherein tilting the support surface away from the actual horizontal position includes tilting the support surface along a lateral axis such that the subject's toes are directed upward.

7. A method according to claim 2, wherein tilting the support surface away from the actual horizontal position includes tilting the support surface along a lateral axis such that the subject's toes are directed downward.

8. A method according any of claims 1-3, wherein tilting the support surface away from the actual horizontal position includes tilting the support surface along a forward directed axis such that the left side of the support surface is directed upward.

9. A method according to any of claims 1-3, wherein tilting the support surface away from the actual horizontal position includes tilting the support surface along a forward directed axis such that the right side of the support surface is directed upward.

10. A method according to any of claims 1-3, wherein tilting the support surface away from the actual horizontal position includes tilting the support surface along an axis other than a lateral or forward facing axis.

11. A method according to claim 2, further comprising:
performing the protocol another time while applying a stimulus to one or both of the subject's legs when having the subject cause repositioning of the support surface; and comparing the actual horizontal position with the horizontal position as it is perceived by the subject; and
comparing results obtained in performing the protocol with and without the stimulus.

12. A method according to claim 11, wherein applying a stimulus to one or both of the subject's legs includes applying a vibratory stimulus.

13. A method according to claim 11, wherein applying a stimulus to one or both of the subject's legs includes applying an electrical stimulus.

14. A method according to claim 2, wherein tilting the support surface away from the actual horizontal position includes tilting the support surface along a lateral axis such that the subject's toes are directed upward and further comprising:
performing the protocol another time while tilting the support surface along a lateral axis such that the subject's toes are directed downward; and
comparing results obtained in performing the protocol with the subject's toes directed upward to results obtained in performing the protocol with the subject's toes directed downward.

15. A method according to claim 2, wherein tilting the support surface away from the actual horizontal position includes tilting the support surface along a lateral axis such that the subject's toes are directed upward and further comprising:
performing the protocol another time while tilting the support surface along a forward directed axis such that the left side of the support surface is directed upward; and
comparing results obtained in performing the protocol with the subject's toes directed downward to results obtained in performing the protocol with the left side of the support surface directed upward.

16. A method according to claim 2, wherein tilting the support surface away from the actual horizontal position includes tilting the support surface along a lateral axis such that the subject's toes are directed upward and further comprising:
performing the protocol another time while tilting the support surface along a forward directed axis such that the right side of the support surface is directed upward; and
comparing results obtained in performing the protocol with the subject's toes directed downward to results obtained in performing the protocol with the right side of the support surface directed upward.

17. A method according to claim 2, wherein tilting the support surface away from the actual horizontal position includes tilting the support surface along a lateral axis such that the subject's toes are directed upward and further comprising:
performing the protocol another time while tilting the support surface along an axis other than a lateral or forward facing axis; and
comparing results obtained in performing the protocol with the subject's toes directed downward to results obtained in performing the protocol with the support surface tilted along an axis other than a lateral or forward facing axis.

18. A method according to any of claims 1-3, further comprising:
comparing the results of the protocol with results of the protocol performed by a defined population of control subjects.

19. A system for assessing a subject's ability to sense orientation using somatosensory inputs derived from contact of the subject with a support surface, the system comprising:
a tiltable support surface;
a harness system for partially assisting the subject in bearing the subject's weight on the support surface; and
a computational device coupled to the support surface for controlling the motion of the support surface and comparing an actual horizontal position with a perceived horizontal position to produce a first result.

20. A system according to claim 19, wherein the computational device compares the first result with a second result derived from a defined population of control subjects.

21. A system according to claim 19, further comprising:
a stimulator coupled for applying stimulus to the subject.

22. A system according to claim 21, wherein the stimulator includes a vibrator for applying vibratory stimulus to the subject.

23. A system according to claim 21, wherein the stimulator includes an electrical stimulator for applying an electrical stimulus to the subject.

24. A system according to claim 21, wherein the stimulator is coupled to the computational device.

25. A system according to claim 19, further comprising:
a stabilizing facility graspable or touchable by the subject, for providing the subject with postural stability.

26. A system according to claim 19, further comprising:
a display device coupled to the computational device.
